(19) United States
(12) Patent Application Publication
(10) Pub. No.: US 2008/0199828 A1
(43) Pub. Date: Aug. 21, 2008

(54) Method of examining irregular defects of dental implant

(75) Inventors: Min-Chun Pan, Ping-Jen City (TW); Han-Bo Zhuang, Dashu Township (TW); Shyh-Yuan Lee, Taipei City (TW)

Correspondence Address: TROXELL LAW OFFICE PLLC, 5205 LEESBURG PIKE, SUITE 1404, FALLS CHURCH, VA, 22041, US

(73) Assignee: National Central University, Taoyuan (TW)

(21) Appl. No.: 11/808,782

(22) Filed: Jun. 12, 2007

(30) Foreign Application Priority Data
Feb. 15, 2007 (TW) 096105803

(51) Int. Cl. A61C 13/00; A61B 5/00; G01N 29/12

(52) U.S. Cl. 433/167; 600/553; 73/649

(57) ABSTRACT

An irregular defect of a dental implant is examined. The dental implant is excited first to obtain vibration responses. The vibration responses is received by accelerometer or sensor to be processed into a resonance frequency diagram. Through the diagram and the position of the accelerometer or sensor, stability of the whole structure and the defect position are diagnosed for evaluation. The present invention can be also applied to implants other than the dental implant.

### FIELD OF THE INVENTION

The present invention relates to examining implant defect; more particularly, relates to evaluating irregular defect of an implant through exciting the implant.

### DESCRIPTION OF THE RELATED ARTS

A first prior art is a U.S. Pat. No. of 5,392,779. The first prior art provides a method and apparatus for the test of an implant attached to a bone of a human or animal subject, where the apparatus comprises a device adapted to be releasably attached to the implant; excitation means for exciting the device with a variable frequency AC excitation signal; and means including a transducer for detecting at least one resonance frequency of the device. The detected resonance frequency is used to assess the degree of attachment of the implant to the bone.

A second prior art is a U.S. Pat. No. of 5,392,779. The second prior art is a dental analyzer, comprising a dental probe for contacting a dental implant; a hammer to impact the dental probe for obtaining vibration responses; and an accelerometer to receive vibration responses from the implant. Through processing the vibration responses, a frequency spectrum is obtained to analyze the stability of the dental implant.

A third prior art is a U.S. Pat. No. of 7,147,467. The third prior art is a tooth mobility measuring apparatus to measure a mobility of a tooth, comprising an impact mechanism to impact the tooth; at least one sensor to detect a displacement state of the tooth; and a tooth mobility calculation mechanism which calculates a tooth mobility of the tooth on the basis of an output signal from the sensor.

The above prior arts measure the stability of the implant through impacting the implant in a non-invasive way. However, only an overall stability of the interface between the implant and an alveolar bone are obtained without indicating specific positions of defect. Not to mention that clinical examination on a 2-dimensional X-ray image film of the implant is insufficient for the diagnosis of irregular bone defects. Hence, the prior arts do not fulfill all users' requests on actual use.

### SUMMARY OF THE INVENTION

The main purpose of the present invention is to provide a non-invasive method for examining and evaluating an osseointegration of the dental implant having an irregular defect.

An other purpose of the present invention is to provide a method for evaluating an irregular defect type through an osseointegration status between the dental implant and an alveolar bone.

The third purpose of the present invention is to obtain more information than by clinical examination based on a 2-dimensional X-ray image film or by measuring resonance frequency of the implant for obtaining stability of the whole structure only.

The fourth purpose of the present invention is to reliably diagnose an implant osseointegration to improve surgeries of dental implant in a rate of success; and to make the diagnosis applicable to implants other than dental implant.

To achieve the above purpose, the present invention is a method of examining an irregular defect of an implant, comp rising steps of: (a) exciting an implant through a vibrating device to obtain vibration responses; (b) obtaining a frequency response diagram with the vibration responses through a spectral analysis; and (c) obtaining a defect status and an osseointegration stability of the implant by referring to at least one position of at least one vibration accelerometer and differences of resonance frequency of the implant. Accordingly, a novel method of examining an irregular defect of an implant is obtained.

### BRIEF DESCRIPTIONS OF THE DRAWINGS

The present invention will be better understood from the following detailed description of the preferred embodiment according to the present invention, taken in con junction with the accompanying drawings, in which

FIG. 1 is the flow view showing the preferred embodiment according to the present FIG. 2 is the schematic view showing the test approach;

FIG. 3A is the view showing the spectrum of the frequency responses at the first vibration accelerometer for no defects;

FIG. 3B is the view showing the spectrum of the frequency responses at the first vibration accelerometer for the defect at the A side;

FIG. 3C is the view showing the spectrum of the frequency responses at the first vibration accelerometer for the defects at both of the A side and the C side;

FIG. 3D is the view showing the spectrum of the frequency responses at the first vibration accelerometer for the serious circular defects;

FIG. 4A is the view showing the spectrum of the frequency responses at the second vibration accelerometer for no defects;

FIG. 4B is the view showing the spectrum of the frequency responses at the second vibration accelerometer for the defect at the A side;

FIG. 4C is the view showing the spectrum of the frequency responses at the second vibration accelerometer for the defects at both of the A side and the C side;

FIG. 4D is the view showing the spectrum of the frequency responses at the second vibration accelerometer for the serious circular defects;

FIG. 5 is the chart view showing the first four resonance frequencies at the first vibration accelerometer;

FIG. 6 is the chart view showing the first four resonance frequencies at the second vibration accelerometer;

FIG. 7 is the chart view showing the resonance frequencies for the defect at the A side;

FIG. 8 is the chart view showing the resonance frequencies for the defects at both of the A side and the C side;

FIG. 9A is the view showing the experimental setup in the status of no defect;

FIG. 9B is the view showing the experimental setup in the status with the A side defect;

FIG. 9C is the view showing the experimental setup in the status with the both A and C side defects; and FIG. 9D is the view showing the experimental setup in the status with the serious circular defects.

### DESCRIPTION OF THE PREFERRED EMBODIMENT

The following description of the preferred embodiment is provided to understand the features and the structures of the present invention.

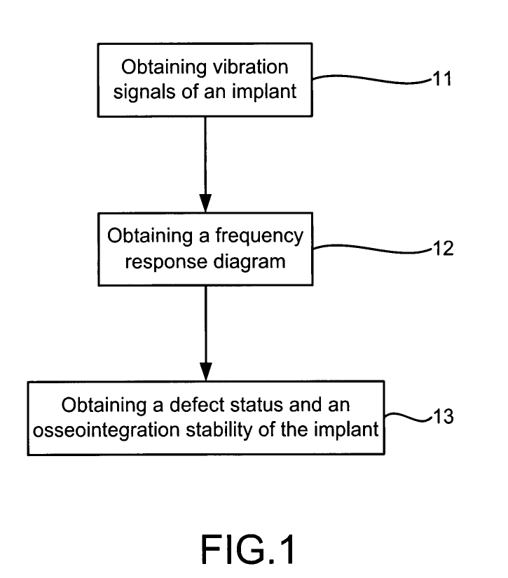

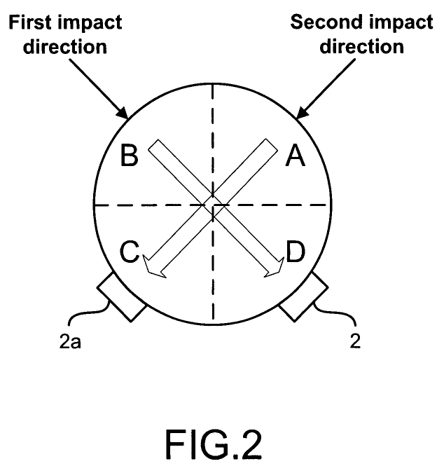

Please refer to FIG. 1, which is a flow view showing a preferred embodiment according to the present invention. As shown in the figure, the present invention is a method of examining an irregular defect of an implant, comprising the following steps:

(a) Obtaining vibration responses of an implant 11: An implant is excited through a vibrating device, like an impact hammer, for obtaining its vibration responses.

(b) Obtaining a frequency response diagram 12: The vibration responses are processed to obtain a frequency response chart through a spectral analysis.

(c) Obtaining a defect status and an osseointegration stability of the implant 13: By referring to at least one position of at least one vibration sensor and differences of resonance frequency of the implant, a defect status and an osseointegration stability of the implant is obtained, where the vibration sensor can be an accelerometer.

Through the above steps, the present invention provides a non-invasive examining method for irregular bone defects from various osseointegration status of a dental implant. According to the dental implant and different structural characteristics of the irregular bone defects, an osseointegration status between the dental implant and an alveolar bone is evaluated quantitatively.

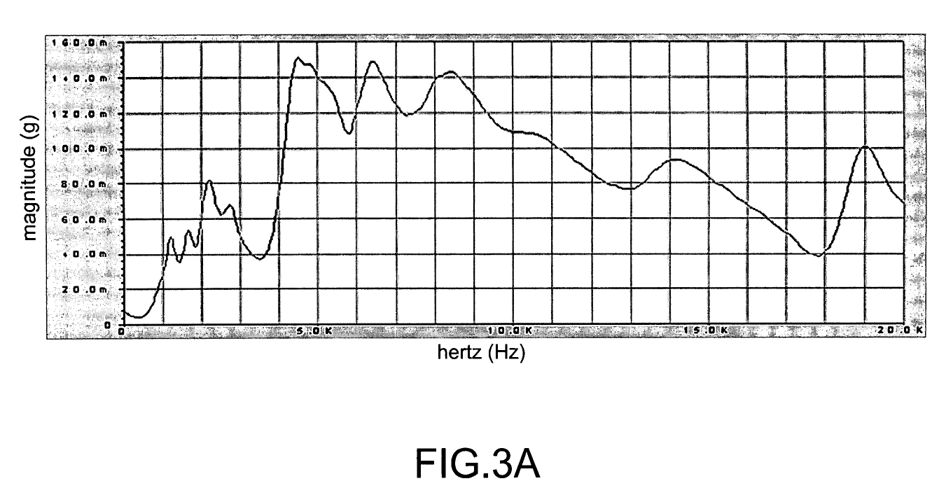

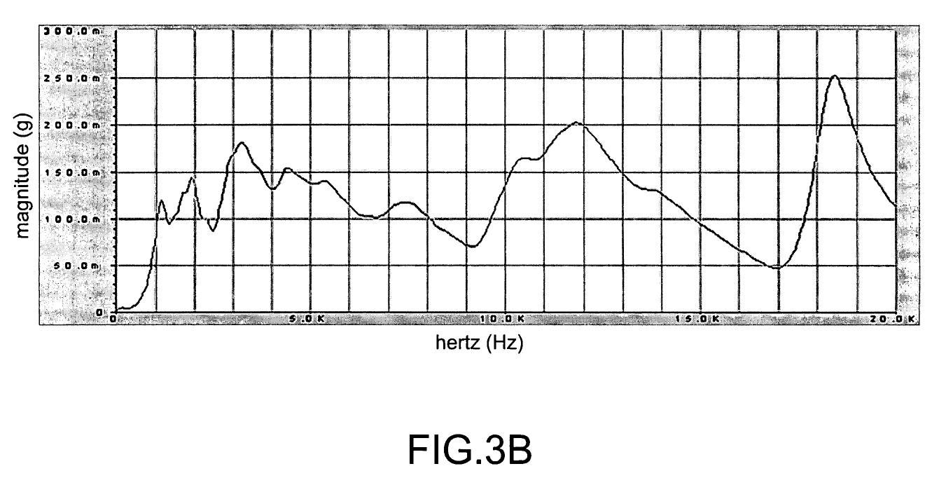

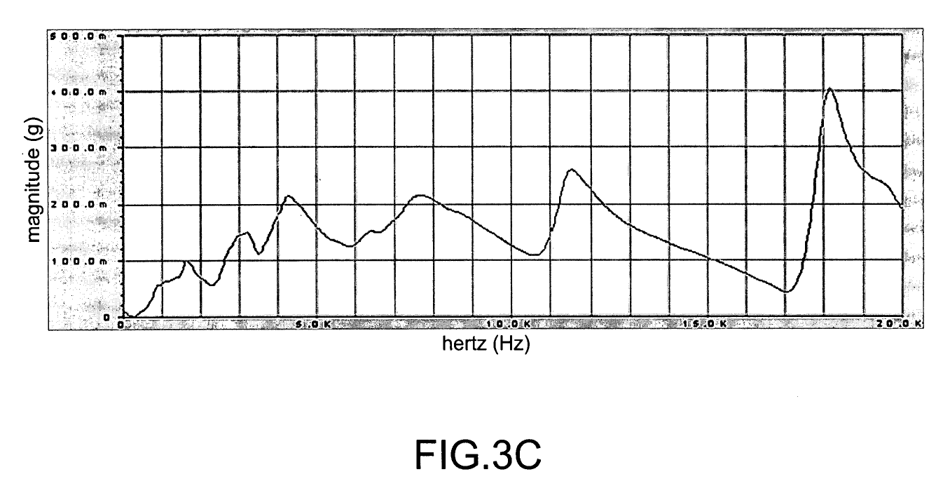

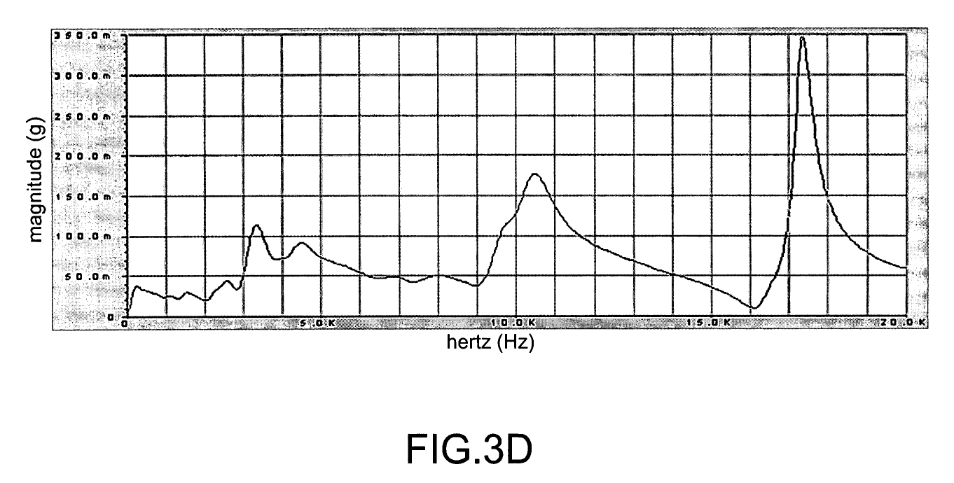

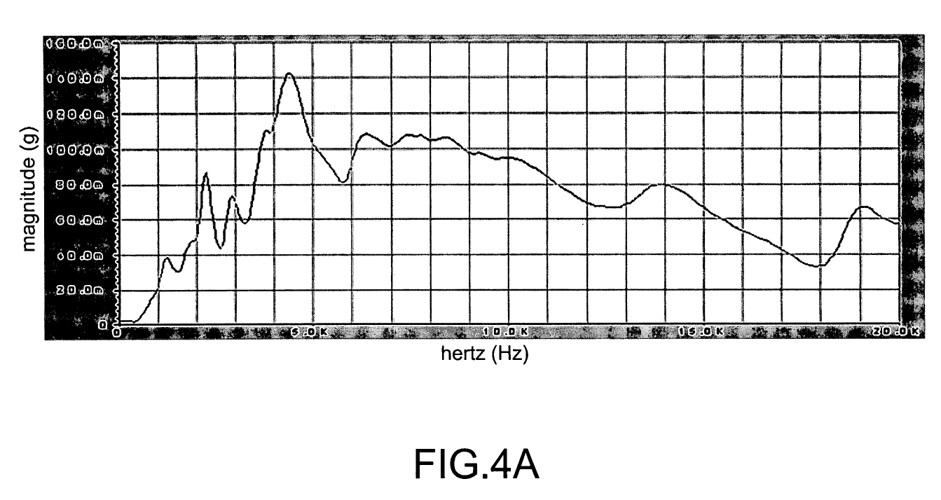

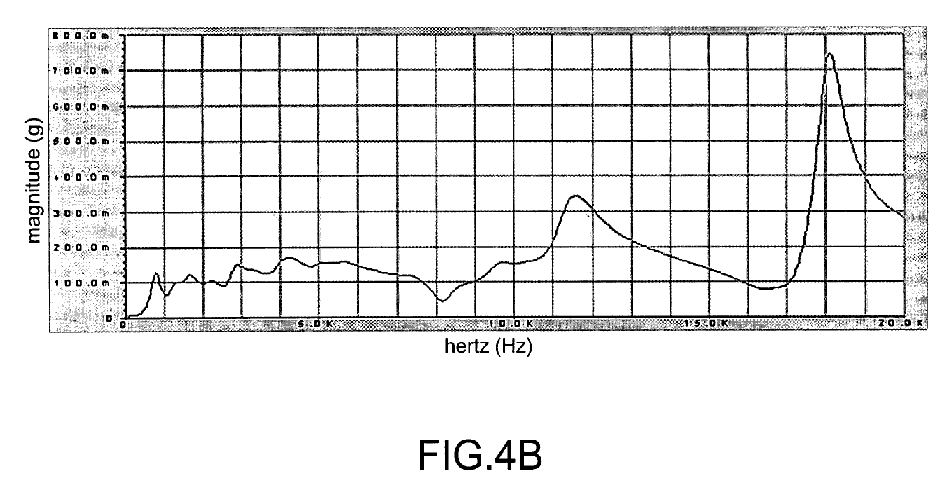

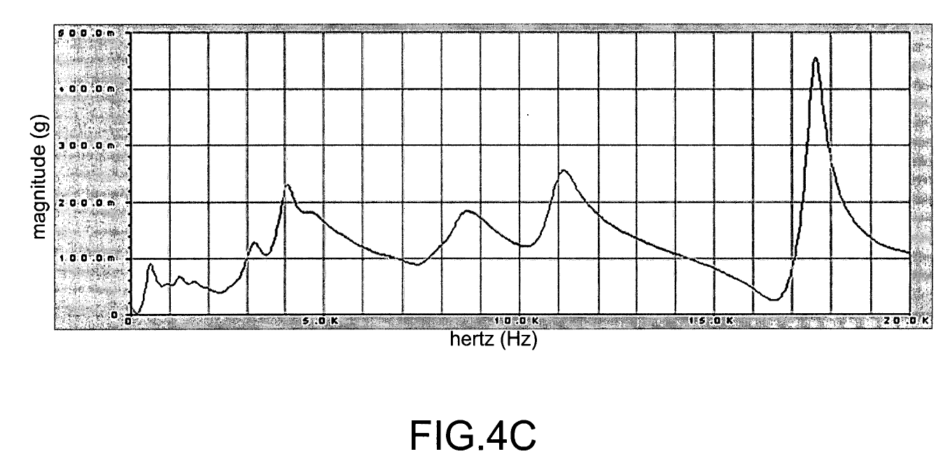

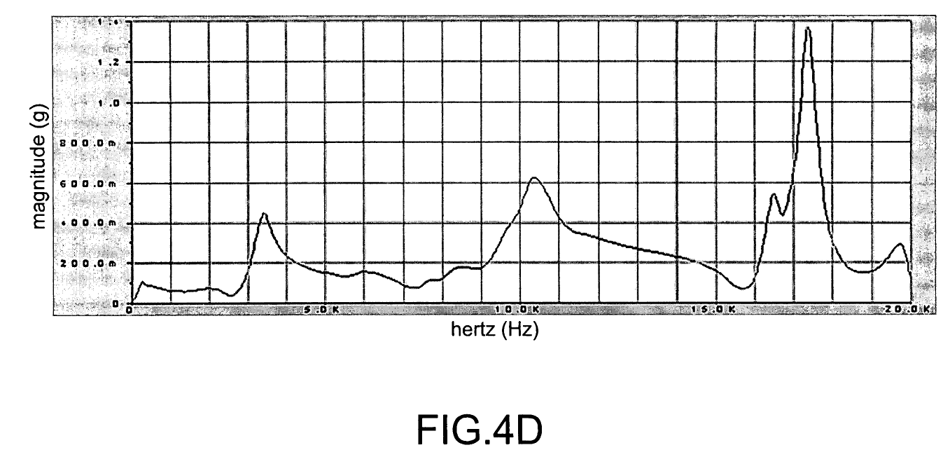

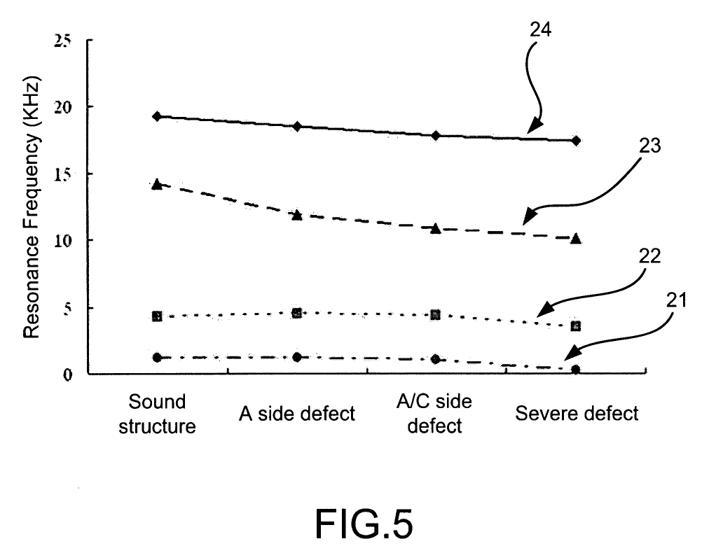

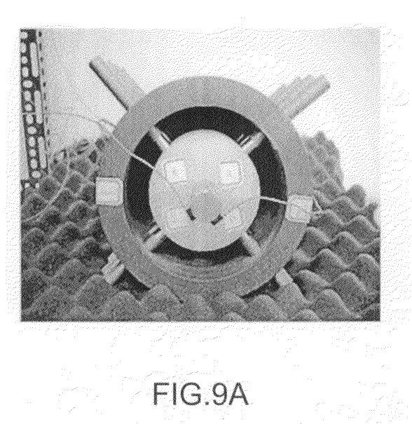

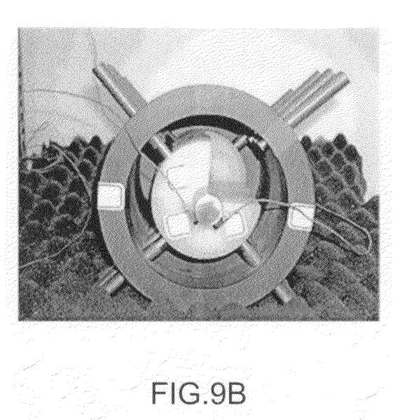

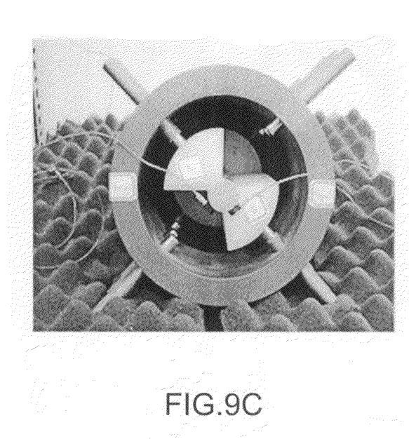

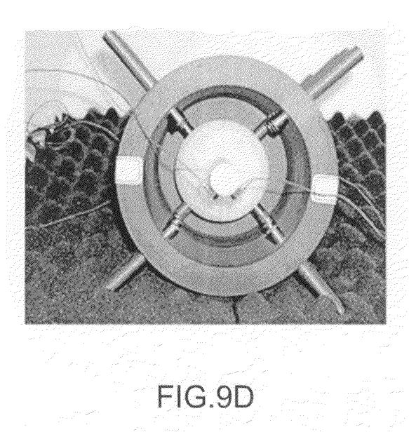

Please refer to FIG. 2, FIG. 3A to FIG. 4D, FIG. 5 to FIG. 8, and 9A to FIG. 9D, which area schematic view showing a test approach; views showing spectrums of frequency responses at a first vibration accelerometer and a second vibration accelerometer for no defects, a defect at A side, defects at both of the A side and C side and serious circular defects; chart views showing the first four resonance frequencies at the first vibration accelerometer and those at the second vibration accelerometer; chart views showing the resonance frequencies for the defect at the A side and the defects at both of the A side and C side; and views showing the experimental setups in the status of no defect, the status with the A side defect, the status with the both A and C side defects and the status with the serious circular defects. As shown in FIG. 9A to FIG. 9D, an enlarged implant is used and four defect statuses are tested. As shown in FIG. 2, a first vibration accelerometer 2 and a second vibration accelerometer 2a are stuck to the implant. The implant is excited with an impact hammer or through any other mechanical force (not shown in the figures). Vibration responses thus obtained are received by the first and the second vibration accelerometers 2, 2a. Then the vibration responses are processed through a spectral analysis to obtain frequency response diagrams, as shown in FIG. 3A to FIG. 4D. Hence, relationships between the resonance frequency of the whole structure and the osseointegration status are obtained.

From values of peak curves on the frequency response diagrams, first four peak values are obtained as indexes. Peak curves are obtained through signals received by the first impact direction and the second impact direction, including first resonance frequency peak curves 21, 21a, second resonance frequency peak curves 22, 22a, third resonance frequency peak curves 23, 23a and fourth resonance frequency peak curves 24, 24a for an implant having no defect, an implant having a defect at an A side, an implant having a defect at the A side and a C side and an implant having a serious defect, respectively. If a structure between a dental implant and an alveolar bone is affected by a defect, a stability of the implant is reduced with a smaller rigidity and, consequently, a resonance frequency of the whole structure is reduced.

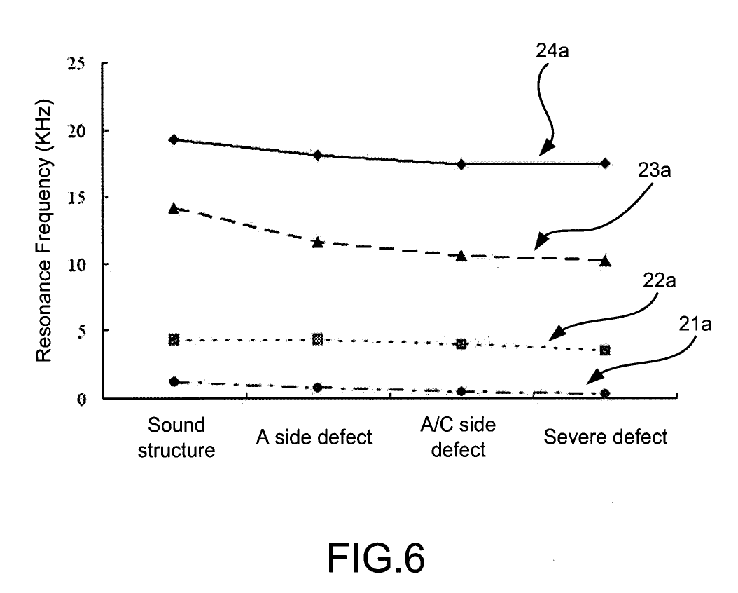

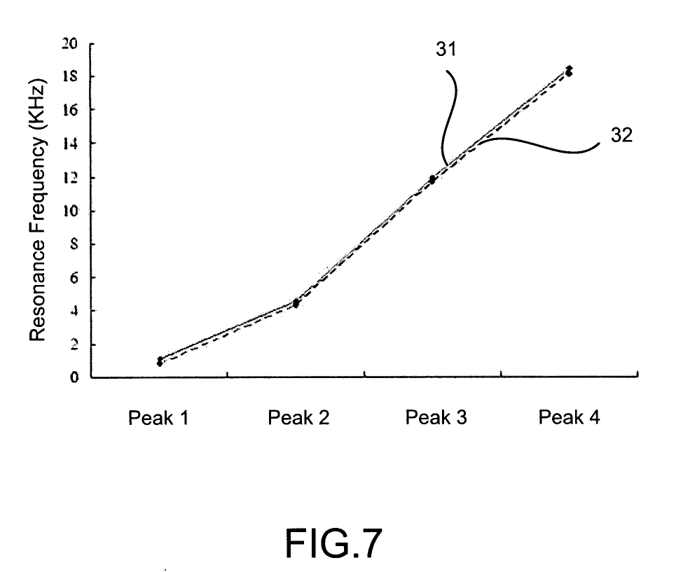

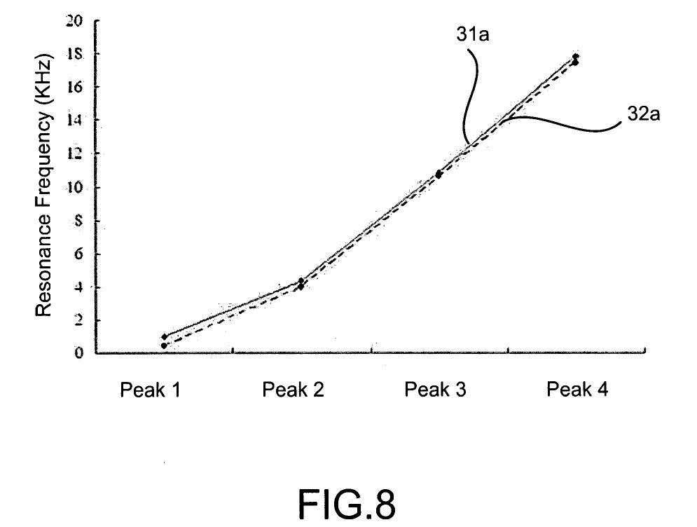

As shown in FIG. 7 and FIG. 8, when impacts are done at a weaker point of the implant having the A side defect and a weaker point of the implant having the A side defect and the C side defect, like a breach or an opening, fifth resonance frequency peak curves 31, 32 are obtained by the first vibration accelerometer and resonance frequency peak curves 31a, 32a are obtained by the second vibration accelerometer. By referring to the above results shown in FIG. 5 and FIG. 6, positions for the defects are thus confirmed. Hence, the present invention is able to figure out defect position.

To sum up, the present invention is a method of examining an irregular defect of an implant, where a better method is provided to evaluate an integration status between an implant and an alveolar bone, better than the insufficient method of clinical examination on a 2-dimensional X-ray image film; and better than the limited method of measuring resonance frequency of an implant for obtaining stability of the whole structure only. By using the present invention, osseointegration diagnosis of an implant is more reliable and thus surgeries of dental implant is improved in a rate of success. Besides, the present invention can be applied to implants other than dental implant too.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

What is claimed is:

1. A method of examining an irregular defect of an implant, comprising steps of:
(a) exciting an implant through a vibrating device to obtain vibration responses;
(b) obtaining a frequency response diagram with said vibration responses through a spectral analysis; and
(c) obtaining a defect status and an osseointegration stability of said implant by referring to at least one position of at least one vibration accelerometer and differences of resonance frequency of said implant.

2. The method according to claim 1,
wherein said vibrating device excites said implant with a mechanical force by an impact hammer

3. The method according to claim 1,
wherein said vibration responses are received by said vibration accelerometer.

4. The method according to claim 1,
wherein said vibration accelerometer is a sensor.

5. The method according to claim 1,
wherein first four peak values among peak values in said frequency response diagram are obtained as indexes of resonance frequency.
